(19) United States
(12) Patent Application Publication
(10) Pub. No.: US 2024/0350713 A1
(43) Pub. Date: Oct. 24, 2024

(54) SYSTEMS FOR SUPPLYING MEDICAL FLUID FOR RENAL REPLACEMENT THERAPY AND METHODS OF OPERATING SUCH SYSTEMS

(71) Applicant: GAMBRO LUNDIA AB, Lund (SE)

(72) Inventors: Olof JANSSON, Vellinge (SE); Michael PETTERSSON, Malmö (SE); Per-Olof BORGQVIST, Lund (SE); Thomas HERTZ, Lund (SE); Jonas FORS, Malmö (SE)

(21) Appl. No.: 18/682,383

(22) PCT Filed: Aug. 8, 2022
(86) PCT No.: PCT/EP2022/072174

§ 371 Date: Feb. 8, 2024

(30) Foreign Application Priority Data
Aug. 9, 2021 (SE) 2150989-8

(51) Int. Cl. A61M 1/16; A61M 1/14

(52) U.S. Cl. CPC A61M 1/1605; A61M 1/155; A61M 1/1565; A61M 2205/3344; A61M 2205/3389; A61M 2205/3393

(57) ABSTRACT

A system comprises a first device and a second device which establish a fluid path between a pump in the first device and a container in the second device through a valve in the second device. A control unit in the second device selectively operates the valve to open the fluid path. The second device is configured to supply a medical fluid by use of a first fluid received from the first device on the fluid path. A control unit in the first device measures fluid pressure in the fluid path and operates to, intermittently during operation of the second device, activate the pump to pump the first fluid into the fluid path and, when the fluid pressure in the fluid path indicates that the valve is closed, deactivate the pump. The first device is operable to replenish the container without being synchronized with the second device.

### TECHNICAL FIELD

The present disclosure relates to the field of renal replacement therapy and in particular to supplying a medical fluid for use in such therapy.

### BACKGROUND ART

Renal replacement therapy (RRT) is a therapy that replaces the normal blood-filtering function of the kidneys. It is used when the kidneys are not working well, which is known as kidney failure and includes acute kidney injury and chronic kidney disease. RRT involves removal of solutes from the blood of a patient suffering from kidney failure, for example by dialysis (hemodialysis, HD, or peritoneal dialysis, PD), hemofiltration, or hemodiafiltration. Depending on modality, RRT may be performed manually or by use of a machine.

In RRT, one or more medical fluids of specific composition are used for treatment of blood. Such medical fluids include so-called dialysis fluid and replacement fluid.

Medical fluids for RRT may be generated by mixing of one or more fluids, for example one or more concentrates with water. In some installations, a separate water preparation device is arranged to receive and process tap water to produce water of sufficient purity and quality, for example by reverse osmosis (RO). The water preparation device is arranged to pump the purified water on demand to a fluid generation device that mixes the purified water with concentrate(s). The fluid generation device may or may not be integrated in a dialysis machine. Dedicated communication interfaces are provided on the water preparation device and the fluid generation device so as to enable the devices to synchronize their operations, for example by exchanging synchronization signals. Such communication interfaces add cost to the water preparation devices and the fluid generation devices and reduce inter-operability between different manufacturers and between old and new equipment.

US2019/0262522 addresses this problem in relation to a fluid generation device which is configured to generate dialysis fluid for PD by use of purified water from a separate water source. US2019/0262522 proposes to provide the water source and the fluid generation device with a respective pump and install a water line between the water source and the fluid generation device to establish fluid communication between the pumps. The fluid generation device operates its pump independently of the pump in the water source. In one of many proposed implementations, the water source is provided with automatic demand control by being configured to continuously monitor the pressure in the water line and control its pump to supply purified water in dependence of the monitored pressure. A similar solution is proposed in US2010/0018923. One drawback of these proposals is that the water source needs to be actively controlled at all times during operation of the fluid generation device. This results in an elevated power consumption and may also over time lead to significant mechanical wear of the pumps in the water source and in the fluid generation device.

The foregoing technical challenges are equally applicable to transfer of medical fluid from a fluid generation device to an RRT machine which is physically separated from the fluid generation device.

### SUMMARY

It is an objective to at least partly overcome one or more limitations of the prior art.

A further objective is to provide a system for supplying a medical fluid for use in RRT while mitigating the need for synchronization between a first device that provides a first fluid and a second device that provides the medical fluid by use of the first fluid.

Another objective is to reduce the power consumption and/or improve the robustness of such a system for supplying medical fluid.

One or more of these objectives, as well as further objectives that may appear from the description below, are at least partly achieved by a system for generating a medical fluid, a system for handling spent medical fluid, and methods according to the independent claims, embodiments thereof being defined by the dependent claims.

A first aspect is a system for supplying a medical fluid for renal replacement therapy. The system comprises a first device which is configured to provide a first fluid. The first device comprises a fluid pump and a first control unit. The system further comprises a second device which is configured to supply the medical fluid by use of the first fluid. The second device comprises a container, a control valve, and a second control unit. The first and second devices are connected to establish a fluid path between the fluid pump in the first device and the container in the second device through the control valve. The second control unit is configured to selectively operate the control valve to open the fluid path. The first control unit is connected to a sensor arrangement which is configured to measure a parameter indicative of fluid pressure in the fluid path. The first control unit is configured to, intermittently during operation of the second device and independent of the parameter, activate the fluid pump to pump the first fluid into the fluid path and, when the parameter indicates that the control valve is closed, deactivate the fluid pump.

A second aspect is a method performed by a first device in fluid communication with a second device that supplies a medical fluid for renal replacement therapy by use of a first fluid. The method comprises: intermittently, while the second device is operated to supply the medical fluid, activating a fluid pump in the first device to pump the first fluid on a fluid path, which extends between the fluid pump in the first device and a container in the second device via a control valve in the second device; obtaining a measurement of a parameter indicative of fluid pressure in the fluid path from a sensor arrangement; and deactivating the fluid pump when the parameter indicates that the control valve is closed.

The first and second aspects applies a trial-and error approach of operating the first device that allows the first device to be operated independently of the second device, while still ensuring that the container in the second device is properly and timely replenished by first fluid from the first device. Further, by only intermittently activating the fluid pump in the first device, the life span of the fluid pump may be extended, and its power consumption reduced. Still further, by having a control valve in the second device to selectively open and close the fluid path between the first and second devices, it is possible to dispense with a pump in the second device for drawing the fluid from the first device. This reduces cost, saves power and increases robustness of the system.

The first and second aspects are applicable to a second device that is configured to generate the medical fluid by processing the first fluid from the first device, for example by adding one or more compounds to the first fluid, and to supply the medical fluid for use in RRT. The first and second aspects are equally applicable to a first device that provides the medical fluid to the second device which is configured to supply the medical fluid for use in RRT. Here, the second device need not process the first fluid to generate the medical fluid since the first fluid constitutes the medical fluid.

A third aspect is a system for handling spent medical fluid from renal replacement therapy. The system comprises a first device comprising a fluid pump and a first control unit. The system further comprises a second device comprising a container arranged to collect spent medical fluid, a control valve and a second control unit. The first and second devices are connected to establish a fluid path between the fluid pump in the first device and the container in the second device through the control valve. The second control unit is configured to selectively operate the control valve to open the fluid path. The first control unit is connected to a sensor arrangement which is configured to measure a parameter indicative of fluid pressure in the fluid path. The first control unit is configured to, intermittently and independent of the parameter, activate the fluid pump to draw the spent medical fluid from the fluid path and, when the parameter indicates that the control valve is closed, deactivate the fluid pump.

A fourth aspect is a method performed by a first device in fluid communication with a second device that provides spent medical fluid from renal replacement therapy. The method comprises: intermittently, while the second device is operated to collect the spent medical fluid in a container in the second device, activating a fluid pump in the first device to draw the spent medical fluid on a fluid path, which extends between the fluid pump in the first device and the container in the second device via a control valve in the second device; obtaining a measurement a parameter indicative of fluid pressure in the fluid path from a sensor arrangement; and deactivating the fluid pump when the parameter indicates that the control valve is closed.

The third and fourth aspects share technical advantages with the first and second aspects, for example that the first device is allowed to operate independently of the second device and that the life span of the fluid pump may be extended and power consumption may be reduced by the intermittent activation of the fluid pump.

A fifth aspect is a computer-readable medium comprising program instructions which, when executed by a processor, cause the processor to perform the method of the second or fourth aspects.

Still other objectives, aspects and advantages, as well as features and embodiments, may appear from the following detailed description, from the attached claims as well as from the drawings.

### BRIEF DESCRIPTION OF THE DRAWINGS

FIGS. 1A-1B are schematic diagrams of example systems in accordance with first and second embodiments, and FIG. 1C is a schematic diagram of a control unit in the systems of FIGS. 1A-1B.

FIG. 2A is a flow chart of an example method of controlling a first device in the systems of FIGS. 1A-1B, FIGS. 2B-2C are flow charts of example methods of controlling a second device in the systems of FIGS. 1A-1B, FIG. 2D is a flow chart of an example method of installing and starting the systems in FIGS. 1A-1B, and FIGS. 2E-2F are timing diagrams of fluid transfer attempts by the first device and fluid admission by the second device in accordance with examples.

FIGS. 3A-3B are flow charts of example methods of controlling a first device in the systems of FIGS. 1A-1B to supply a first fluid to a second device and to receive spent medical fluid from the second device, respectively.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments.

It will furthermore be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing the scope of the present disclosure. As used herein, the terms “multiple”, “plural” and “plurality” are intended to imply provision of two or more elements. The term “and/or” includes any and all combinations of one or more of the associated listed elements.

Well-known functions or structures may not be described in detail for brevity and/or clarity. Unless otherwise defined, all terms (including technical and scientific terms) used herein have the same meaning as commonly understood by one of ordinary skill in the art to which this disclosure belongs.

Embodiments relate to systems for supplying a medical fluid for use in renal replacement therapy (RRT). The systems comprise a first device and a second device. The first device is configured to provide a first fluid to the second device through a fluid path that extends between the first and second devices. The second device is configured to provide, based on the first fluid, a medical fluid for use in RRT. The second device may or may not be configured to also perform RRT by use of the medical fluid. In other words, the second device may be an RRT machine, for example a dialysis machine. Embodiments described herein presume that the second device comprises a container that is arranged to receive the incoming first fluid on the fluid path from the first device, and that the second device gradually consumes the first fluid in the container as it provides the medical fluid for use in RRT. Since the first fluid in the container is consumed, the second device needs to replenish the container by receiving first fluid on the fluid path. Embodiments described herein further presume that the container in the second device is not replenished continuously but that the first fluid is only intermittently admitted into the container by the second device selectively opening an inlet valve, for example whenever the amount of first fluid in the container reaches a predefined minimum value.

The embodiments to be described in the following serve to allow the first device to be operated independently of the second device, while still ensuring that the container in the second device is properly and timely replenished.

In some embodiments, this technical effect is achieved by providing the first device with a sensor arrangement which is configured to, directly or indirectly, measure the fluid pressure in the fluid path between the first and second devices, and by appropriately configuring a control unit of the first device to operate based on the fluid pressure measured by the sensor arrangement. Specifically, the control unit is configured to, intermittently during operation of the second device, activate a fluid pump (“supply pump”) in the first device to pump the first fluid into the fluid path and, when the fluid pressure in the fluid path indicates that the inlet value of the second device is closed, deactivate the fluid pump. In these embodiments, the first device thus applies a trial-and-error (TAE) approach by repeatedly attempting to push the first fluid on the fluid path into the container of the second device. These attempts are made independently of the fluid pressure in the fluid path, for example in accordance with a predefined time schedule. If the inlet valve on the second device is closed at such an attempt, the fluid pressure in the fluid path will rapidly increase, given that the first fluid is an incompressible liquid. The increase in fluid pressure is detected by the first device, which thereby terminates the attempt. Should the inlet valve on the second device be open at such an attempt, the first device will continue to pump the first fluid into the container of the second device, either in accordance with a predefined setting or until the second device closes the inlet valve. By the TAE approach, the first device is rendered operatively independent of the second device, while enabling the container in the second device to be timely replenished with first fluid from the first device. At the same time, by the intermittent activation, power consumption will be limited. Further, at least for some types of fluid pumps, wear is limited by the intermittent activation compared to continuous activation.

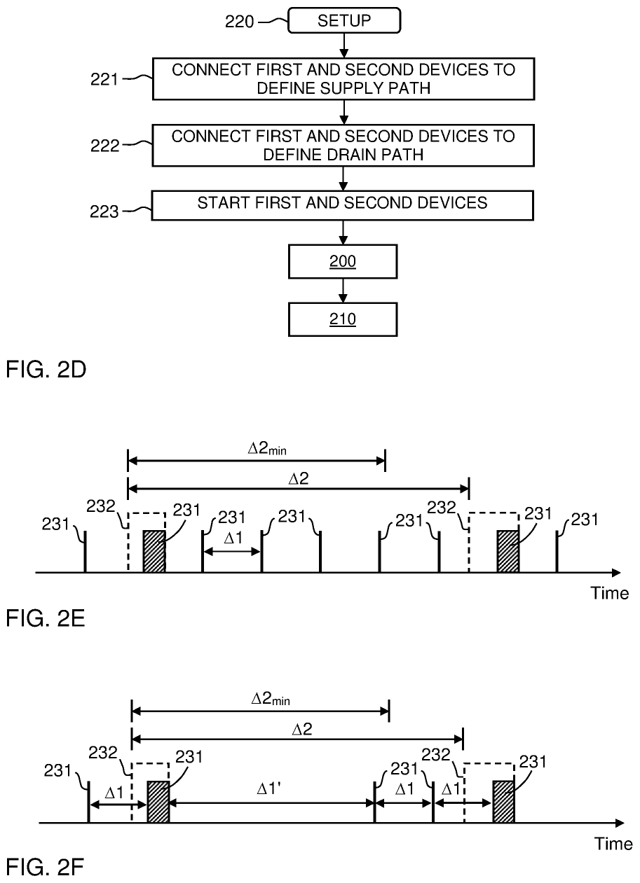

As noted, the fluid transfer attempts arc triggered independently of the fluid pressure in the fluid path between the first and second device. In some embodiments, the time interval between the fluid transfer attempts by the first device may be set in relation to an expected time difference between replenishments of the container in the second device (“replenishment interval”) and/or an expected time from opening of the inlet valve until complete depletion of the container (“time to depletion”). For example, the second device may by design have a maximum (largest possible) supply rate of medical fluid. The maximum supply rate of the medical fluid corresponds to a maximum consumption rate of the first fluid in the container and thus to a minimum (smallest possible) replenishment interval. Likewise, the maximum supply rate corresponds to a minimum time to depletion, given by the remaining amount of first fluid in the container when the inlet valve is opened divided by the maximum consumption rate. Examples of criteria for setting the time interval between fluid transfer attempts are given below with reference to FIGS. 2E-2F.

The TEA approach may be applied in a similar manner when the first device is arranged to receive a fluid (“second fluid”) from the second device on a second fluid path. By analogy with the foregoing embodiments, it is presumed that the second device comprises a container for collecting the second fluid and an outlet valve that is intermittently opened to establish fluid communication between the container and the second fluid path. To enable the TEA approach, the first device has a sensor arrangement which is configured to, directly or indirectly, measure the fluid pressure in the second fluid path, and the control unit of the first device is configured to operate based on this fluid pressure. Specifically, in some embodiments, the control unit is configured to, intermittently during operation of the second device, activate a fluid pump (“drain pump”) in the first device to draw the second fluid into the second fluid path and, when the fluid pressure in the second fluid path indicates that the outlet value of the second device is closed, deactivate the fluid pump. Thus, the first device repeatedly attempts to draw the second fluid on the second fluid path from the second device. These attempts are made independently of the fluid pressure in the second fluid path, for example in accordance with a predefined time schedule. In some embodiments, the second fluid is spent medical fluid, which is produced when the above-mentioned medical fluid is used in RRT. The first device may be configured to direct the incoming spent medical fluid to a drain or collect the spent medical fluid in a container associated with the first device. In another alternative, the first device may be configured to produce (“regenerate”) medical fluid from the spent medical fluid.

Embodiments are applicable to any type of RRT, including by not limited to hemodialysis, hemofiltration, hemodiafiltration and peritoneal dialysis.

In some embodiments, the second device is a dialysis machine for hemodialysis, hemofiltration or hemodiafiltration. Such a dialysis machine may be dedicated to treatment of patients with acute kidney injury (AKI), commonly known as “acute dialysis”. Treatment of AKI by hemodialysis is typically performed continuously, by so-called Continuous Renal Replacement Therapy (CRRT). Dialysis machines for acute dialysis commonly comprise scales, on which containers or “fluid bags” are releasably arranged. The operation of the dialysis machine is controlled based on the weight of the fluid bags, given by the readings of the scales. Commonly, at least one of the fluid bags is arranged to hold a dialysis fluid, which is used in the dialysis treatment, and at least one of the fluid bags is arranged to receive spent dialysis fluid. As is well-known to the skilled person, the dialysis treatment involves extracting excess fluid from the patient, commonly known as “ultrafiltrate”. The ultrafiltrate is included in the spent dialysis fluid. The amount of ultrafiltrate extracted from the patient and the rate of extraction are important treatment parameters during dialysis. During operation, the dialysis machine calculates and monitors these treatment parameters based on the readings of the scales, which represent the weights of the fluid bags.

Conventionally, dialysis fluid for acute dialysis is supplied in prefilled fluid bags, which are hung on one of more scales of the dialysis machine. Therefore, acute dialysis is taxing on the caretaker, who needs to repeatedly replace a fluid bag that is running empty for a new prefilled fluid bag. An alternative would be to generate the dialysis fluid on site.

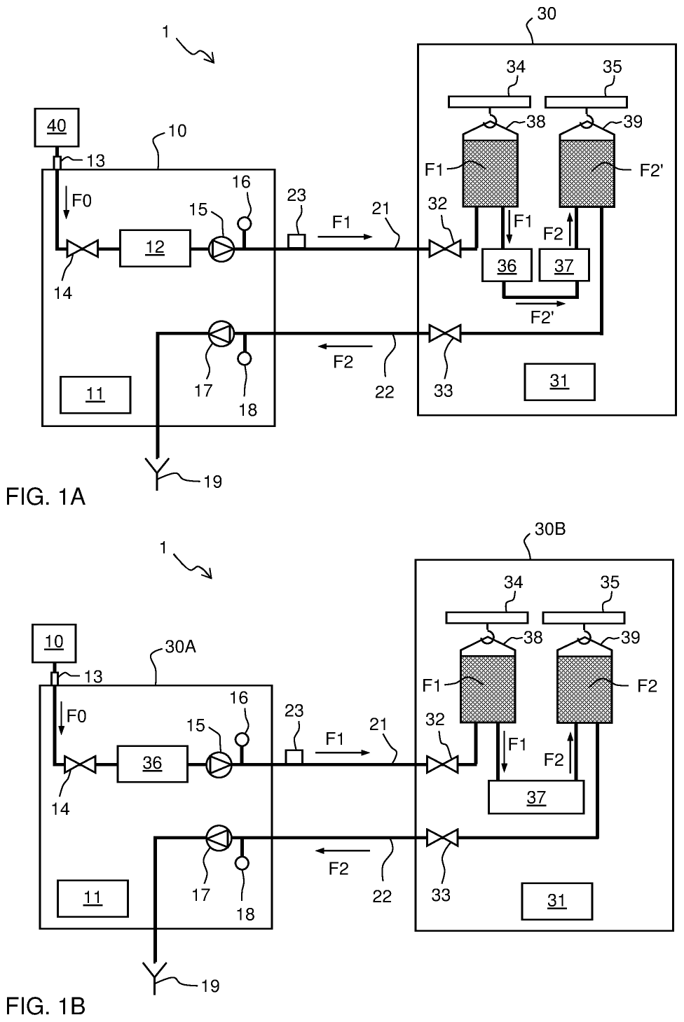

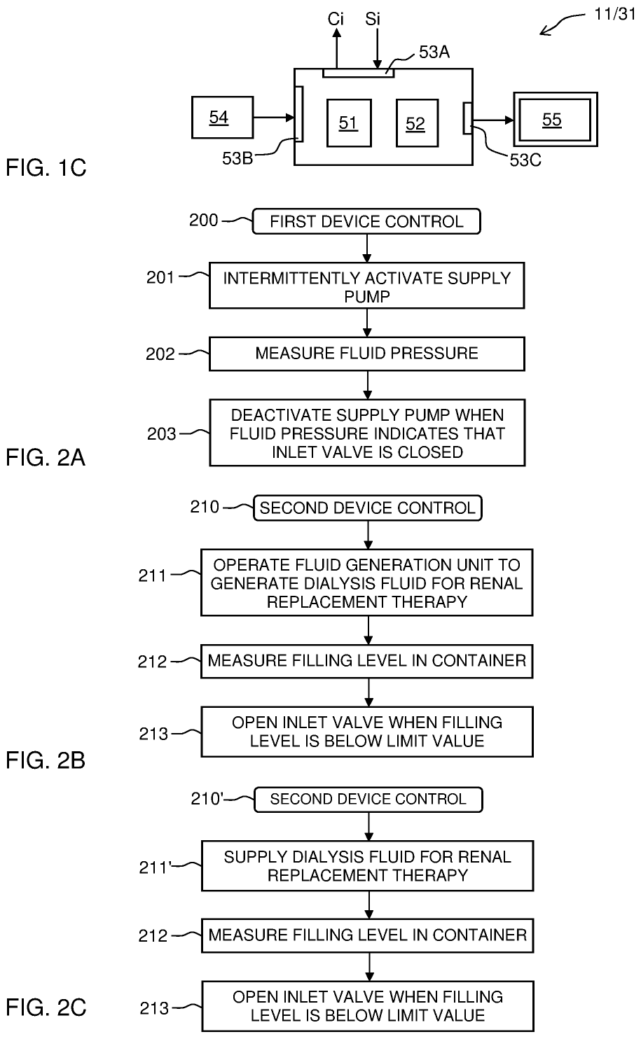

In one proposal, the dialysis machine is arranged to receive dialysis fluid from a separate fluid preparation device, which may be configured to prepare dialysis fluid from tap water or purified water. Thereby, an empty fluid bag on a scale of an existing dialysis machine for acute dialysis may be connected to receive dialysis fluid from the fluid preparation device. This fluid bag may need to be replenished or refilled with dialysis fluid during operation of the dialysis machine. However, the process of refilling of the fluid bag may affect the operation of the dialysis machine and should therefore be performed rarely and during a confined time period. With reference to the foregoing discussion about first and second devices, it is realized that the first device may be a fluid preparation device and the second device may be a dialysis machine. A corresponding implementation example is described below with reference to FIG. 1B.

In another proposal, the dialysis machine is capable of generating dialysis fluid from purified water, for example by mixing one or more concentrates or substances with the purified water. The purified water may be received from a separate water purification device, which may be configured to process tap water into purified water. In the example of a dialysis machine for acute dialysis, the purified water may be received in a fluid bag on a scale of the dialysis machine, and the mixing may be performed in this fluid bag and/or downstream thereof. Like in the foregoing proposal, it may be desirable to perform the refilling of the fluid bag rarely and during a confined time period. Thus, the first device may be a water preparation device and the second device may be a dialysis machine. A corresponding implementation example is described below with reference to FIG. 1A.

FIG. 1A schematically illustrates a treatment system 1 that includes a water preparation device, WPD, 10 (“first device”) and an RRT machine 30 (“second device”) which are connected for fluid communication. A control unit 11 is arranged to control the operation of the WPD 10, and a control unit 31 is arranged to control the operation of the RRT machine 30. The WPD 10 comprises a water processing unit 12, a connector 13 for connecting the WPD 10 to a source 40 of tap water, an inlet valve 14, a supply pump 15, a first pressure sensor 16, a drain pump 17, and a second pressure sensor 18. The control unit 11 is operable to open the inlet valve 14 to admit tap water F0 from the source 40 into the water processing unit 12, which is configured to process the tap water into purified water suitable for dialysis. The water processing unit 12 may be of conventional construction and may perform one or more heating, filtering, softening, reverse osmosis (RO), deionization (DI), UV irradiation, distillation, etc. The purified water may thereby be generated to comply with thresholds for both chemical purity and microbiologic and endotoxin purity. The supply pump 15 is arranged to pump the purified water F1 on a first fluid path (“supply path”) 21 to the RRT machine 30, and the pressure sensor 16 is arranged to measure the fluid pressure in the supply path 21. In the illustrated example, the WPD 10 is also configured to receive spent dialysis fluid F2 from the RRT machine 30 on a second fluid path (“drain path”) 22. The fluid paths 21, 22 may be defined by flexible tubing and/or channels in a cassette and may be permanently or releasably connected to the WPD 10 and the RRT machine 30. The drain pump 17 is arranged to pump spent dialysis fluid from the drain path 22 to a drain 19 or into a container (not shown), and the pressure sensor 18 is arranged to measure the fluid pressure in drain path 22. The pressure sensors 16, 18 may be seen to define a pressure sensor arrangement in the WPD 10.

In an alternative implementation of the system 1 in FIG. 1A, the source 40 is configured to supply at least partly purified water. In such an alternative, the water processing unit 12 may be simplified or even omitted.

The RRT machine 30 comprises an inlet valve 32, an outlet valve 33, a first scale 34, a second scale 35, a fluid generation unit 36, a treatment unit 37, a first fluid bag 38, and a second fluid bag 39. The first fluid bag 38 is arranged on the scale 34, and the second fluid bag 39 is arranged on the second scale 35. The control unit 31 is operable to open the inlet valve 32 to admit purified water F1 from the WPD 10 on the supply path 21 into the fluid bag 38 for intermediate storage. Purified water F1 is transferred from the fluid bag 38 into the fluid generation unit 36, which is configured to generate a dialysis fluid F2′ from the purified water F1. As noted above, the fluid generation unit 36 may be configured to mix one or more concentrates or substances (powder or liquid) with the purified water to form the dialysis fluid. The dialysis fluid F2′ is supplied to the treatment unit 37, which configured to perform the dialysis treatment of the blood of a patient (not shown). The treatment unit 37 is configured to transfer spent dialysis fluid F2 to the fluid bag 39 for intermediate storage. In some embodiments, the reading of the scale 34 is used by the control unit 31 to determine the amount of dialysis fluid F2′ supplied to the treatment unit 37, and the reading of the scale 35 is used by the control unit 31 to determine the amount of spent dialysis fluid F2 provided by the dialysis treatment, for calculation of one or more treatment parameters related to ultrafiltration. The control unit 31 is operable to open the outlet valve 33 to allow spent dialysis fluid F2 to be drawn into the WPD 10 on the drain path 22. The dialysis treatment as such is well-known to the skilled person and will not be described herein. The skilled person also understands that the RRT machine 30 may be partly formed by disposables, which are discarded after a dialysis treatment. Such disposables may include the fluid bags 38, 39, at least part of the fluid generation unit 36, and at least part of the treatment unit 37. For example, it is conventional practice that a disposable line set and a disposable dialyzer are installed on an RRT machine 30 to define a dialysis fluid circuit and an extracorporeal blood circuit in the treatment unit 37. It may also be noted that the pressure sensors 16, 18 may be included in the disposable and connected for signal transfer to the WPD 10 when the disposable is installed. The disposable may also include the fluid paths 21, 22. In some embodiments, the valves 32, 33 are pinch valves.

FIG. 1B is a schematic illustration of a treatment system 1 that includes a fluid preparation device, FPD, 30A (“first device”) and an RRT machine 30B (“second device”) which are connected for fluid communication. The FPD 30A is configured to generate and supply dialysis fluid F1 to the RRT machine 30 and to receive spent dialysis fluid F2 from the RRT machine 30. The following description will focus on differences over the system in FIG. 1A. The FPD 30A is principally similar to the WPD 10, in that it comprises a control unit 11, a connector 13, an inlet valve 14, a supply pump 15, a first pressure sensor 16, a drain pump 17, and a second pressure sensor 18. However, compared to the WPD 10, the FPD 30A is connected by connector 13 to a source 10 of purified water F0 and comprises a fluid generation unit 36 configured to generate the dialysis fluid F1. The fluid generation unit 36 may correspond to the fluid generation unit 36 in the RRT machine 30 in FIG. 1A but may be of different construction. Generally, the fluid generation unit 36 in the FPD 30A may be configured to mix one or more concentrates or substances with the purified water F0 to form the dialysis fluid F1.

The RRT machine 30B is principally similar to the RRT machine 30 in FIG. 1A in that it comprises a control unit 31, an inlet valve 32, an outlet valve 33, a first scale 34, a second scale 35, a treatment unit 37, a first fluid bag 38, and a second fluid bag 39. However, there is no functional equivalent of the fluid generation unit 36 in the RRT machine 30B. The control unit 31 is operable to open the inlet valve 32 to admit dialysis fluid F1 from the FPD 30A on the supply path 21 into the fluid bag 38 for intermediate storage. Dialysis fluid F1 is transferred from the fluid bag 38 to the treatment unit 37, which may be identical to the treatment unit 37 in FIG. 1A. The treatment unit 37 is configured to transfer spent dialysis fluid F2 to the fluid bag 39 for intermediate storage. In some embodiments, the reading of the scale 34 is used by the control unit 31 to determine the amount of dialysis fluid F1 supplied to the treatment unit 37, and the reading of the scale 35 is used by the control unit 31 to determine the amount of spent dialysis fluid F2 provided by the dialysis treatment, for calculation of one or more treatment parameters related to ultrafiltration. The control unit 31 is operable to open the outlet valve 33 to allow spent dialysis fluid F2 to be drawn into the FPD 30A on the drain path 22.

FIG. 1C is a schematic block diagram of the respective control unit 11, 31 in FIGS. 1A-1B. The control unit 11, 31 is configured to generate control signals Ci for controlling the operation of the first/second device in accordance with a control program comprising computer instructions. The control program may also be configured to operate based on input signals Si received by the control unit 11, 31. In the WPD 10, the control unit 11 may be connected to provide control signals for the valve 14, the water processing unit 12 and the pumps 15, 17, and to receive input signals from the pressure sensors 16, 18. In the RRT machine 30, the control unit 31 may be connected to provide control signals for the valves 32, 33, the fluid generation unit 36 and the treatment unit 37, and to receive input signals from the scales 34, 35. In the FPD 30A, the control unit 11 may be connected to provide control signals for the valve 14, the fluid generation unit 36 and the pumps 15, 17, and to receive input signals from the pressure sensors 16, 18. In the RRT machine 30B, the control unit 31 may be connected to provide control signals for the valves 32, 33 and the treatment unit 37, and to receive input signals from the scales 34, 35. The foregoing is merely a simplified example and the respective control unit 11, 31 may be configured to generate further control signals Ci and receive further input signals Si, as readily appreciated by the skilled person.

The respective control unit 11, 31 comprises a processor 51 and computer memory 52. The control program is stored in the memory 52 and executed by the processor 51. The control program may be supplied to the control unit 11, 31 on a computer-readable medium, which may be a tangible (non-transitory) product (e.g., magnetic medium, optical disk, read-only memory, flash memory, etc.) or by a propagating signal. In the illustrated example, the control unit 11, 31 comprises a signal interface 53A for providing control signals Ci and receiving input signals Si. In the illustrated example, the control unit 11, 31 also comprises an input interface 53B for connection to one or more input devices 54 that enable an operator to input control data, as well as an output interface 53C for connection to one or more output devices 55 for providing feedback data to the operator. For example, the input device(s) 54 may comprise a keyboard, keypad, computer mouse, control button, touch screen, printer, microphone, etc., and the output device(s) 55 may comprise a display device, a touch screen, an indicator lamp, an alarm device, a speaker, etc.

FIG. 2A is a flow chart of a method 200 for controlling the first device 10, 30A in the systems 1 shown in FIG. 1A and FIG. 1B. The method 200 may be performed by the control unit 11. In step 201, the supply pump 15 is intermittently activated to pump the fluid F1 that is provided by the first device 10, 30A into the supply path 21 and towards the second device 30, 30B. Step 202 is performed during step 201 and involves measuring the fluid pressure in the supply path 21, for example in a measurement signal from the pressure sensor 16. In step 203, the supply pump 15 is deactivated when the fluid pressure indicates that the inlet valve 32 of the second device 30, 30B is closed, for example if the fluid pressure exceeds a limit value. Thereby, the first device 10, 30A implements the above-mentioned TAE approach to provide the fluid F1 to the second device 30, 30B.

It is understood that the fluid F1 will be pumped into the container 38 if the inlet valve 32 is open when the supply pump 15 is activated by step 201. In some embodiments, the supply pump 15 will be remain active until the measured fluid pressure indicates that the inlet valve 32 is closed. It is also conceivable to control the activation based on a predefined setting, for example a maximum limit for the duration of the activation of the supply pump 15 or the amount of fluid pumped during an activation. For example, the supply pump 15 may be automatically deactivated when such a maximum limit exceeded even if the fluid pressure indicates that the inlet valve 32 is still open. Such an automatic deactivation may be indicative of an operational error in the first or second device and the first device may generate an alert or alarm for the operator, for example on the output device 55 in FIG. 1C.

The pump 15 may need to be stopped quickly by step 203 if the inlet valve 32 is closed when the pump 15 is activated, to avoid build-up of excessive fluid pressure potentially causing leaks in the supply path 21 or at its connection to the first and second devices. To mitigate the risk for excessive fluid pressure and enable the use of a simpler pump 15, a compliance arrangement 23 may be arranged in fluid communication with the supply path 21, as exemplified in FIGS. 1A-1B. The compliance arrangement 23 is configured to absorb part of the pressure increase in the supply path 21. The compliance arrangement 23 may be an expandable tubing portion or chamber, which is arranged anywhere between the supply pump 15 and the inlet valve 32. Alternatively or additionally, the compliance arrangement 23 may be included in the pump 15. For example, the pump 15 may be configured to leak fluid opposite to the pumping direction (known as “back slip”) when pressure builds up downstream of the pump 15. In some embodiments, the pump 15 has a selected characteristic of back slip as a function of pressure. Non-limiting examples include a peristaltic pump configured with a selected degree of occlusion, or a gear pump. Alternatively or additionally, build-up of excessive fluid pressure in the supply path 21 is mitigated by configuring the inlet valve 32 with a soft-closing function. The soft-closing function may be implemented by operating and/or configuring the inlet valve 32 to close slowly. In this context, “slowly” implies that the response time of the inlet valve 32 is controlled in relation to the stopping time of the pump 15 to ensure that the fluid pressure in the supply path 21 is below a limit value, which is specific to the design of the supply path 15. The response time is the time required to switch the inlet valve 32 from a fully open state to a fully closed state, and the stopping time is the time required to bring the running pump 15 to a halt. In some embodiments, the inlet valve 32 has a response time of 1-10 seconds.

In some embodiments, the pressure sensor 16 is or comprises a pressure switch, which is configured to indicate when the fluid pressure exceeds a configurable limit pressure. Step 203 may infer the state of the inlet valve 32 from a signal generated by the pressure switch (cf. Si in FIG. 1C). Presence of the indication in the signal infers that the inlet valve 32 is closed, and absence of the indication infers that the inlet valve 32 is open.

FIG. 2B is a flow chart of a method 210 for controlling the second device 30 in the system 1 of FIG. 1A. The method 210 may be performed by the control unit 31. In step 211, the fluid generation unit 36 is operated to generate dialysis fluid F2′ from the purified water F1 in the container 38 and to supply the dialysis fluid F2′ to the treatment unit 37. In some embodiments, step 211 may be arranged for so-called “online generation” of dialysis fluid, which implies that dialysis fluid is generated at a rate that matches the consumption of the dialysis fluid by the treatment unit 37. In other words, dialysis fluid is generated on demand for use by the treatment unit 37. Step 212 is performed during step 211 and involves measuring the filling level in the container 38. In FIG. 1A, the filling level may be inferred from the weight measured by scale 34. When the filling level falls below a lower limit value, step 213 opens the inlet valve 32. Step 213 may keep the inlet valve 32 open until the filling level in the container 38 reaches an upper limit value, whereupon the inlet value 32 is closed and the container 38 has been replenished with purified water F1. Since the first device 10 performs method 200 while the second device 30 performs method 210, the container 38 will be replenished without synchronization between the first and second devices 10, 30.

FIG. 2C is a flow chart of a method 210′ for controlling the second device 30B in the system 1 of FIG. 1B. The method 210′ may be performed by the control unit 31. In step 211′, the second device 30B is operated to supply dialysis fluid F1 from the container 38 to the treatment unit 37, which is operated to perform the dialysis treatment. For example, the dialysis fluid 31 may be drawn from the container 38 by a pump (not shown) in the treatment unit 37. Steps 212-213 of method 210′ may be identical to steps 212-213 of method 210.

The first device 10, 30A may be controlled obtain the spent dialysis fluid from the second device 30, 30B by a modification of the method 200 in FIG. 2A. In correspondence with step 201, the drain pump 17 is intermittently activated to draw the fluid F2 on the drain path 22 from the second device 30, 30B. In correspondence with step 202, the fluid pressure is measured in the drain path 22, for example in a measurement signal from the pressure sensor 18. In correspondence with step 203, the drain pump 17 is deactivated when the fluid pressure indicates that the outlet valve 33 of the second device 30, 30B is closed, for example if the fluid pressure is below a limit value. Thereby, the first device 10, 30A implements the above-mentioned TAE approach to obtain the fluid F2 from the second device 30, 30B.

Similarly, the second device 30, 30B may be controlled by a modification of the method 210. In correspondence with step 212, the filling level in the container 39 is measured while the dialysis treatment is performed by the treatment unit 37 (cf. step 211). The filling level may be inferred from the weight measured by scale 35. In correspondence with step 213, when the filling level exceeds an upper limit value, the outlet valve 33 is opened to establish fluid communication between the container 39 and the drain path 22.

FIG. 2D is a flow chart of a method 220 of setting up a dialysis treatment by use of the first and second devices in FIGS. 1A-1B. In step 221, the first and second devices are connected by installation of the supply path 21. In step 222, the first and second devices are connected by installation of the drain path 22. For example, the paths 21, 22 may be defined in a disposable that is installed on the first and second devices. In step 223, the first and second devices are started, whereupon the first device operates in accordance with method 200 and the second device operates in accordance with method 210 or 210′. Further, the first device may operate in accordance with the above-mentioned modification of method 200 to remove spent dialysis fluid from the second device, and the second device may operate in accordance with the above-mentioned modification of method 210 to selectively open the drain path 22.

FIGS. 2E-2F show two examples of the timing of the activation of the supply pump 15 in the first device and the opening of the inlet valve 32 in the second device.

In FIG. 2E, the supply pump 15 is activated at regular time intervals, Δ1 (cf. step 201 in FIG. 2A). Each activation (“replenishment attempt”) is represented as 231. The inlet valve 32, on the other hand, is opened at a time point when the amount of fluid F1 in container 38 falls below the lower limit value (cf. step 213 in FIGS. 2B-2C), and this time point may vary over time. In FIG. 2E, the time periods when the inlet valve 32 is open are represented by dashed lines 232. In the following, these time periods are denoted “open periods”. As seen in FIG. 2E, when the activation 231 occurs during an open period 232, the activation 231 continues until the inlet valve 32 closes. As noted above, the second device may be associated with a minimum replenishment interval which is given by constraints of the second device. In FIG. 2E, the minimum replenishment interval is indicated as Δ2ₘᵢₙ, and the actual time difference between two consecutive open periods 232 is indicated as Δ2. To ensure continuous operation of the second device, Δ1 may be set to be smaller than the above-mentioned time to depletion for the container 38. Further, Δ1 may be set to be smaller than Δ2ₘᵢₙ. For example, Δ1 may be set so that there are multiple activations 231 within Δ2ₘᵢₙ, as exemplified in FIG. 2E.

In FIG. 2F, the first device postpones the next activation 231 for a time period Δ1′ when an activation has resulted in a replenishment of the container 38. After Δ1′, the first device may resume activations 231 of the supply pump at regular time intervals, Δ1. The time period Δ1′ may be set in relation to Δ2ₘᵢₙ, to ensure that no open period 232 occurs during Δ1′. In one example, Δ1′ is set not to exceed Δ2ₘᵢₙ-Δ1.

In a variant of the timing diagram in FIG. 2F, the time interval Δ1 between activations 231 may be varied according to any suitable function. In one example, the first device may decrease Δ1 over time until an activation 231 results in a replenishment of the container 38.

In a further variant, the control unit 11 of the first device 10, 30A comprises a function that adjusts the time interval Δ1 between the activations 231 based on the timing of preceding open periods 232. Thereby, the control unit 11 is configured to learn the operation of the second device 30, 30B, for example by use of a machine learning-based function. The machine learning-based function may be re-initiated for each treatment session or be performed continuously over a plurality of treatment sessions for the same or different patients.

In all examples described in the foregoing, the activation of the supply pump 15 is performed in accordance with a time schedule, which may be predefined (including regular time intervals and/or variable time intervals according to a predefined function) or be dynamically determined by machine learning.

The timing diagrams in FIGS. 2E-2F and the associated description, as well as the machine learning variant, are equally applicable to the activation of the drain pump 17 and the opening of the outlet valve 33.

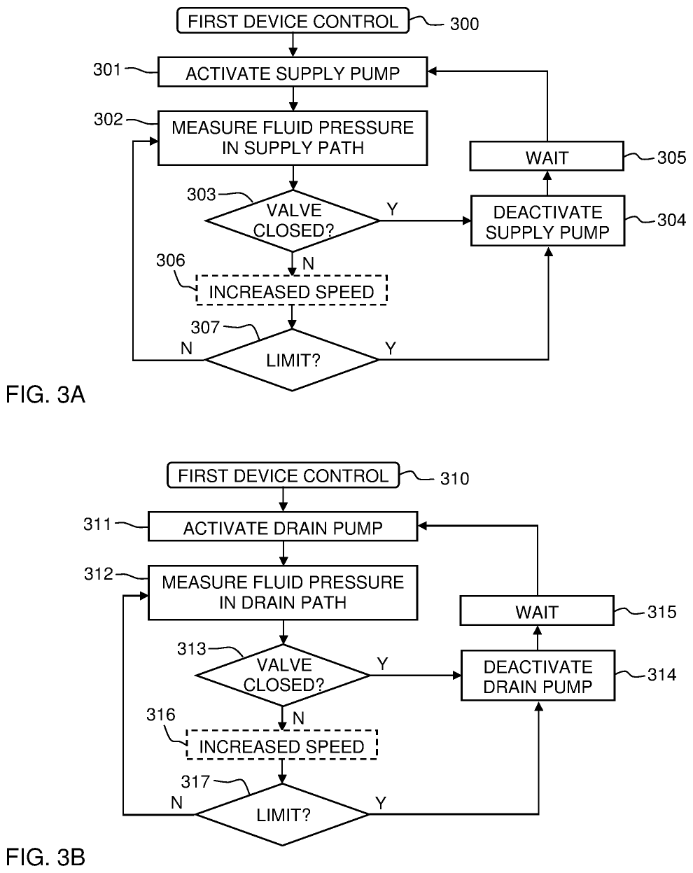

FIG. 3A is a flow chart of a further example method 300 for controlling a first device to provide a fluid to a second device. For clarity, the method 300 will be described with reference to the systems 1 in FIGS. 1A-1B. In step 301, the supply pump 15 is activated. In step 302, the fluid pressure in the supply path 21 is measured or monitored. If step 303 infers from the fluid pressure that the inlet value 32 is closed, the method 300 proceeds to step 304, which deactivates the supply pump 15. Then, step 305 halts the method 300 for a waiting period before proceeding to step 301, in which the supply pump 15 is again activated. If step 303 infers from the fluid pressure that the inlet valve 32 is open, the method 300 may proceed to step 307 which may evaluate if the above-mentioned maximum limit (time or amount) is exceeded. If the maximum limit is not exceeded, step 307 proceeds to step 302 and the supply pump 15 remains activated. On the other hand, if the maximum limit is exceeded, step 307 proceeds to step 304, which deactivates the supply pump 15 and optionally discontinues the method 300 and generates an alert for the operator. In a variant, step 307 is omitted. As indicated by dashed lines, the method 300 may comprise a step 306 which increases the speed of the supply pump 15 according to any suitable function over time. By step 306, it is possible to start the supply pump 15 at a low pumping speed to reduce risk of building up an excessive pressure in the supply path before step 303 detects that inlet valve 32 is closed and the supply pump 15 is stopped after deactivation by step 304. Reverting to step 305, the waiting period may be a fixed time period, for example to implement the regular time intervals Δ1 in FIG. 2E. In a variant, step 305 may apply different waiting periods for different conditions. For example, the waiting period may be set to Δ1′ if the container 38 is deemed have been replenished and may otherwise be set to Δ1.

FIG. 3B is a flow chart of a corresponding method 310 for controlling a first device to extract a fluid from a second device. For clarity, the method 310 will be described with reference to the systems 1 in FIGS. 1A-1B. In step 311, the drain pump 17 is activated. In step 312, the fluid pressure in the drain path 22 is measured or monitored. If step 313 infers from the fluid pressure that the outlet value 33 is closed, the method 310 proceeds to step 314, which deactivates the drain pump 17. Then, step 315 halts the method 310 for a waiting period before proceeding to step 311, in which the drain pump 17 is again activated. Like step 305 in FIG. 3A, step 315 may use a fixed waiting period or apply different waiting periods for different conditions. If step 313 infers from the fluid pressure that the outlet valve 33 is open, the method 310 may proceed to step 317 which may evaluate if a maximum limit (time or amount) is exceeded. If the maximum limit is not exceeded, step 317 proceeds to step 312 and the drain pump 17 remains activated. On the other hand, if the maximum limit is exceeded, step 317 proceeds to step 314, which deactivates the drain pump 17 and optionally discontinues the method 310 and generates an alert for the operator. In a variant, step 317 is omitted. As indicated by dashed lines, the method 310 may comprise a step 316 which increases the speed of the drain pump 17 in correspondence with step 306 in the method 300.

The systems in FIGS. 1A-1B are only given as examples. Embodiments are generally applicable to any system for supplying medical fluid for use in RRT, in which any type of first fluid is to be pumped from a first device to a second device which is configured to supply a medical fluid based on the first fluid. The second device may be configured to perform RRT, as in the systems of FIGS. 1A-1B. Alternatively, the second device may be arranged to supply the medical fluid to a separate device that performs RRT. The medical fluid may be any fluid that is consumed during RRT. For example, instead of dialysis fluid, the medical fluid may be a replacement fluid used in convective therapy such as hemofiltration or hemodiafiltration. It is realized that the first device is configured to supply the medical fluid at a quality suitable for its use during RRT.

Further, the filling level of the respective container 38, 39 may be determined by other means than scales, for example by a level sensor, which may be arranged for continuous level sensing or point level sensing, including but not limited to a pneumatic sensor, a conductive sensor, a probe-based sensor, a float-based sensor, an optical sensor, or an ultrasonic sensor.

It should be noted that any type of sensor arrangement may be installed to sense, directly or indirectly, the pressure in the fluid paths 21, 22. Thus, the respective pressure sensor disclosed herein may be replaced by another sensor that is configured to measure a parameter indicative of fluid pressure. In one example, fluid pressure in the fluid path 21, 22 is indirectly given by a power-consumption parameter of the respective pump 15, 17. For example, the power-consumption parameter may represent a drive current and/or a drive voltage of the pump 15, 17. In another example, at least for some types of pumps, fluid pressure may be indirectly given by the speed of the pump 15, 17.

It may also be noted that the extraction of spent medical fluid from the second device by the first device may be omitted. For example, the spent medical fluid may be pumped directly to a drain or to a storage container by the second device.

While the subject of the present disclosure has been described in connection with what is presently considered to be the most practical and preferred embodiments, it is to be understood that the subject of the present disclosure is not to be limited to the disclosed embodiments, but on the contrary, is intended to cover various modifications and equivalent arrangements included within the spirit and the scope of the appended claims.

In the following, a set of clauses are recited to summarize some aspects and embodiments of the invention as disclosed in the foregoing.
C1. A system for supplying a medical fluid (F2′; F1) for renal replacement therapy, said system comprising: a first device (10; 30A) which is configured to provide a first fluid (F1), the first device (10) comprising a fluid pump (15) and a first control unit (11); a second device (30; 30B) which is configured to supply the medical fluid (F2′; F1) by use of the first fluid (F1), the second device (30; 30B) comprising a container (38), a control valve (32), and a second control unit (31); wherein the first and second devices are connected to establish a fluid path (21) between the fluid pump (15) in the first device (10) and the container (38) in the second device (30; 30B) through the control valve (32); wherein the second control unit (31) is configured to selectively operate the control valve (32) to open the fluid path (21); wherein the first control unit (11) is connected to a sensor arrangement (16, 18) which is configured to measure a parameter indicative of fluid pressure in the fluid path (21); and wherein the first control unit (11) is configured to, intermittently during operation of the second device (30; 30B) and independent of the parameter, activate the fluid pump (15) to pump the first fluid (F1) into the fluid path (21) and, when the parameter indicates that the control valve (32) is closed, deactivate the fluid pump (15).
C2. The system of C1, wherein the first control unit (11) is configured to activate the fluid pump (15) in accordance with a time schedule.
C3. The system of C1 or C2, wherein the first control unit (11) is configured to activate the fluid pump (15) at regular time intervals (Δ1).
C4. The system of any preceding clause, wherein the first control unit (11) is configured to separate consecutive activations of the fluid pump (15) by a time interval that is less than an expected time to depletion of the container (38) when the fluid path (21) is opened by the second control unit (31).
C5. The system of any preceding clause, wherein the first control unit (11) is configured to postpone activation of the fluid pump for a waiting period (Δ1′) after a transfer of the first fluid (F1) into the container (38) by the first device (10; 30A), wherein the waiting period (Δ1′) is set in relation to an expected time interval (Δ2ₘᵢₙ ) between consecutive openings of the fluid path (21) by the second control unit (31).
C6. The system of any preceding clause, wherein the second control unit (31) is configured to determine a filling level in the container (38) and open the control valve (32) when the filling level is below a limit value.
C7. The system of C6, wherein the second device (31) further comprises a scale (34) arranged to measure a weight of the container (38), and wherein the second control unit (31) is connected to the scale (34) and is configured to determine the filling level based on the weight measured by the scale (34).
C8. The system of any preceding clause, wherein the first control unit (11) is configured to, after activating the fluid pump (15) and when the parameter indicates that the control valve (32) is open, operate the fluid pump (15) in accordance with a predefined setting and/or until the parameter indicates that the control valve (32) is closed.
C9. The system of any preceding clause, wherein the first control unit (11) is configured to activate the fluid pump (15) to operate at a first speed and, when parameter indicates that the control valve (32) is open, operate the fluid pump (15) at a second speed that is higher than the first speed.
C10. The system of any preceding clause, wherein the fluid path (21) comprises a compliance arrangement (23), which is configured to absorb part of an increase in the fluid pressure resulting from an activation of the fluid pump (15) when the control valve (32) is closed.
C11. The system of any preceding clause, wherein a response time of the control valve (32) is controlled in relation to a stopping time of the fluid pump (15) to maintain the fluid pressure below a limit value.
C12. The system of any preceding clause, wherein the sensor arrangement (16, 18) comprises a pressure switch which is configured to signal when the fluid pressure exceeds a limit pressure.
C13. The system of any preceding clause, wherein the first fluid (F1) is purified water.
C14. The system of any one of C1 -C12, wherein the first fluid (F1) is the medical fluid.
C15. The system of any preceding clause, wherein the medical fluid (F2′; F1) is a dialysis fluid.
C16. The system of any preceding clause, wherein the second device (30; 30B) is a dialysis machine.
C17. The system of C16, wherein the second device (30; 30B) is a dialysis machine for treatment of acute kidney injury, AKI.
C18. The system of any preceding clause, wherein the second device (30; 30B) comprises a second container (39) which is arranged to hold spent medical fluid (F2), and a second control valve (33), wherein the first device (11) comprises a second fluid pump (17), wherein the first and second devices (10, 30; 30A, 30B) are connected to establish a second fluid path (22) between the second fluid pump (17) in the first device (10) and the second container (39) in the second device (30; 30B) through the second control valve (33), wherein the second control unit (31) is configured to selectively operate the second control valve (33) to open the second fluid path (22), wherein the sensor arrangement (16, 18) is further configured to measure a further parameter indicative of fluid pressure in the second fluid path (22), and wherein the first control unit (11) is configured to, intermittently during operation of the second device (30; 30B) and independent of the further parameter, activate the second fluid pump (17) to draw the spent medical fluid (F2) from the second fluid path (22) and, when the further parameter indicates that the second control valve (33) is closed, deactivate the second fluid pump (17).
C19. A system for handling spent medical fluid (F2) from renal replacement therapy, said system comprising: a first device (10; 30A) comprising a fluid pump (17) and a first control unit (11); a second device (30; 30B) comprising a container (39) arranged to collect spent medical fluid (F2), a control valve (33) and a second control unit (31); wherein the first and second devices (10, 30; 30A, 30B) are connected to establish a fluid path (22) between the fluid pump (17) in the first device (10; 30A) and the container (39) in the second device (30; 30B) through the control valve (33); wherein the second control unit (31) is configured to selectively operate the control valve (33) to open the fluid path (22); wherein the first control unit (11) is connected to a sensor arrangement (16, 18) which is configured to measure a parameter indicative of fluid pressure in the fluid path (22); and wherein the first control unit (11) is configured to, intermittently and independent of the parameter, activate the fluid pump (17) to draw the spent medical fluid (F2) from the fluid path (22) and, when the parameter indicates that the control valve (33) is closed, deactivate the fluid pump (17).
C20. A method performed by a first device (10; 30A) in fluid communication with a second device (30; 30B) that supplies a medical fluid (F2′; F1) for renal replacement therapy by use of a first fluid (F1), said method comprising: intermittently, while the second device is operated to supply the medical fluid, activating (201) a fluid pump in the first device to pump the first fluid on a fluid path, which extends between the fluid pump in the first device and a container in the second device via a control valve in the second device; obtaining (202) a measurement of a parameter indicative of fluid pressure in the fluid path from a sensor arrangement; and deactivating (203) the fluid pump when the parameter indicates that the control valve is closed.
C21. A method performed by a first device (10; 30A) in fluid communication with a second device (30; 30B) that provides spent medical fluid (F2) from renal replacement therapy, said method comprising: intermittently, while the second device is operated to collect the spent medical fluid in a container in the second device, activating (211) a fluid pump in the first device to draw the spent medical fluid on a fluid path, which extends between the fluid pump in the first device and the container in the second device via a control valve in the second device; obtaining (212) a measurement of a parameter indicative of fluid pressure in the fluid path from a sensor arrangement; and deactivating (213) the fluid pump when the parameter indicates that the control valve is closed.
C22. A computer-readable medium comprising program instructions which, when executed by a processor (51), cause the processor (51) to perform the method of clause C20 or C21.

1. A system for supplying a medical fluid for renal replacement therapy, the system comprising:
a first device configured to provide a first fluid, the first device comprising a fluid pump and a first control unit; and
a second device configured to supply the medical fluid by use of the first fluid, the second device comprising a container, a control valve, and a second control unit,
wherein the first and second devices are connected to establish a fluid path between the fluid pump in the first device and the container in the second device through the control valve,
wherein the second control unit is configured to selectively operate the control valve to open the fluid path,
wherein the first control unit is connected to a sensor arrangement that is configured to measure a parameter indicative of fluid pressure in the fluid path, and
wherein the first control unit is configured to, intermittently during operation of the second device and independent of the parameter, activate the fluid pump to pump the first fluid into the fluid path and, when the parameter indicates that the control valve is closed, deactivate the fluid pump.

2. The system of claim 1, wherein the first control unit is configured to activate the fluid pump in accordance with a time schedule.

3. The system of claim 1, wherein the first control unit is configured to activate the fluid pump at regular time intervals.

4. The system of claim 1, wherein the first control unit is configured to separate consecutive activations of the fluid pump by a time interval that is less than an expected time to depletion of the container when the fluid path is opened by the second control unit.

5. The system of claim 1, wherein the first control unit is configured to postpone activation of the fluid pump for a waiting period after a transfer of the first fluid into the container by the first device, wherein the waiting period is set in relation to an expected time interval between consecutive openings of the fluid path by the second control unit.

6. The system of claim 1, wherein the second control unit is configured to determine a filling level in the container and open the control valve when the filling level is below a limit value.

7. The system of claim 6, wherein the second device further comprises a scale arranged to measure a weight of the container, and wherein the second control unit is connected to the scale and is configured to determine the filling level based on the weight measured by the scale.

8. The system of claim 1, wherein the first control unit is configured to, after activating the fluid pump and when the parameter indicates that the control valve is open, operate the fluid pump in accordance with a predefined setting and/or until the parameter indicates that the control valve is closed.

9. The system of claim 1, wherein the first control unit is configured to activate the fluid pump to operate at a first speed and, when parameter indicates that the control valve is open, operate the fluid pump at a second speed that is higher than the first speed.

10. The system of claim 1, wherein the fluid path comprises a compliance arrangement, which is configured to absorb part of an increase in the fluid pressure resulting from an activation of the fluid pump when the control valve is closed.

11. The system of claim 1, wherein a response time of the control valve is controlled in relation to a stopping time of the fluid pump to maintain the fluid pressure below a limit value.

12. The system of claim 1, wherein the sensor arrangement comprises a pressure switch which is configured to signal when the fluid pressure exceeds a limit pressure.

13. The system of claim 1, wherein the first fluid is purified water.

14. The system of claim 12, wherein the first fluid is the medical fluid.

15. The system of claim 1, wherein the medical fluid is a dialysis fluid.

16. The system of claim 1, wherein the second device is a dialysis machine.

17. The system of claim 16, wherein the second device is a dialysis machine for treatment of acute kidney injury.

18. The system of claim 1, wherein the second device comprises a second container which is arranged to hold spent medical fluid, and a second control valve, wherein the first device comprises a second fluid pump, wherein the first and second devices are connected to establish a second fluid path between the second fluid pump in the first device and the second container in the second device through the second control valve, wherein the second control unit is configured to selectively operate the second control valve to open the second fluid path, wherein the sensor arrangement is further configured to measure a further parameter indicative of fluid pressure in the second fluid path, and wherein the first control unit is configured to, intermittently during operation of the second device and independent of the further parameter, activate the second fluid pump to draw the spent medical fluid from the second fluid path and, when the further parameter indicates that the second control valve is closed, deactivate the second fluid pump.

19. A system for handling spent medical fluid from renal replacement therapy, the system comprising:
a first device comprising a fluid pump ) and a first control unit; and
a second device comprising a container arranged to collect spent medical fluid, a control valve and a second control unit,
wherein the first and second devices are connected to establish a fluid path between the fluid pump in the first device and the container in the second device through the control valve,
wherein the second control unit is configured to selectively operate the control valve to open the fluid path,
wherein the first control unit is connected to a sensor arrangement which that is configured to measure a parameter indicative of fluid pressure in the fluid path, and
wherein the first control unit is configured to, intermittently and independent of the parameter, activate the fluid pump to draw the spent medical fluid from the fluid path and, when the parameter indicates that the control valve is closed, deactivate the fluid pump.

20. A method performed by a first device in fluid communication with a second device that supplies a medical fluid for renal replacement therapy by use of a first fluid, the method comprising:
intermittently, while the second device is operated to supply the medical fluid, activating a fluid pump in the first device to pump the first fluid on a fluid path, which extends between the fluid pump in the first device and a container in the second device via a control valve in the second device;
obtaining a measurement of a parameter indicative of fluid pressure in the fluid path from a sensor arrangement; and
deactivating the fluid pump when the parameter indicates that the control valve is closed.

21. A method performed by a first device in fluid communication with a second device that provides spent medical fluid from renal replacement therapy, the method comprising:
intermittently, while the second device is operated to collect the spent medical fluid in a container in the second device, activating a fluid pump in the first device to draw the spent medical fluid on a fluid path, which extends between the fluid pump in the first device and the container in the second device via a control valve in the second device;
obtaining a measurement of a parameter indicative of fluid pressure in the fluid path from a sensor arrangement; and
deactivating the fluid pump when the parameter indicates that the control valve is closed.

22. A computer-readable medium comprising program instructions which, when executed by a processor, cause the processor to perform the method of claim 2.
